(19) United States
(12) Patent Application Publication
(10) Pub. No.: US 2026/0053401 A1
(43) Pub. Date: Feb. 26, 2026

(54) HYDROGEL BALL

(71) Applicants: Brian Michael Coyle, Canyon, CA (US); Alys Larsen, Canyon, CA (US)

(72) Inventors: Brian Michael Coyle, Canyon, CA (US); Alys Larsen, Canyon, CA (US)

(21) Appl. No.: 19/377,546

(22) Filed: Nov. 3, 2025

(51) Int. Cl. A61B 5/1473; A61B 5/00; A61B 5/145; A61B 10/00

(52) U.S. Cl. CPC A61B 5/14735; A61B 5/14507; A61B 5/14546; A61B 5/6861; A61B 10/0051

(57) ABSTRACT

A Hydrogel Ball includes a flexible housing with a hydrogel inside that retains, expels, and absorbs fluid through holes in the housing. The hydrogel can be modified to bind biomarkers in absorbed fluid, and release them on command. The Hydrogel Ball is configured for use in the oral cavity of an animal to capture, collect, concentrate, and release salivary and gingival biomarkers.

### RELATED APPLICATIONS

The present application claims the benefit of U.S. Provisional Patent Application No. 63/873,959 filed Sep. 2, 2025 and titled “Device and Method for Sampling Canine Oral Fluid for Detection of Matrix Metalloproteinases”, the contents of which are incorporated in this disclosure by reference in their entirety.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### I. Field of the Invention

The present invention relates to methods and devices for collecting biological samples from mammals, and more particularly to a method and device for sampling saliva that uses a superabsorbent hydrogel that emits and absorbs fluid, and the hydrogel may be modified to bind specific molecules in absorbed fluid.

### II. Description of the Related Art

Test strips and absorbent pads are often utilized to collect biological specimens for further analysis. Medical swabs have been comprised of an elongated shaft with a fibrous (foam or cotton) tip, or a serrated metal tip to collect culture samples from various body areas, including the mouth, ear, nose, or throat. Test strips have been comprised of plastic or paper coated with antigens, antibodies, or drugs that bind a molecule of interest on the test strip.

Test strips are widely used to collect samples. One such system was disclosed in U.S. Pat. No. 5,260,194 to Olson (P1) which is directed toward a method and device for determining the presence of an antigen or drug which specifically binds to an antibody contained on a test strip.

Sample collection systems are configured with absorbent pads and analysis reagents in a single device. One such system was disclosed in U.S. Pat. No. 8,025,851 to Slowey & Giddings (P2) which is directed toward a saliva sampling device that contains one or more absorbent pads in contact with a lateral flow analyte test strip, for collecting adequate fluid samples and enabling rapid test result reporting.

U.S. Pat. No. 10,119,968 to Lansing (P3) describes a collection system configured with a movable test strip container unit and reagent capsule. The test strip container has a test strip inside. After sample collection the test strip is moved into a reagent capsule for analysis.

Besides test strips and swabs, saliva has been collected using mouth rinses and saliva expectoration methods. Mouth rinse methods instruct patients to rinse their mouth with distilled water for 20 or 30 seconds, then expectorate the sample into a container. Collection with saliva expectoration typically has patients tilt their heads down to pool saliva in the mouth for around 30 seconds, then expectorate into a container. Another saliva expectoratio method stimulates saliva with chewing. Patients chew on a material, such as a piece of parafilm, for around 30 seconds, which stimulates saliva production, then expectorate into a container.

Direct comparison of sample collection with test strips, swabs, whole mouth rinse, or saliva expectoration is difficult. Test strips and swabs are not comfortably used for extended periods. A few seconds of swabbing may not collect the same levels of biomarkers as rinses or saliva expectoration, if the biomarkers are released gradually over more than a few seconds in the mouth. Oral rinses are obtained from a diluted saliva extract (a mixture of the rinsing solution and collected saliva) with an unknown dilution factor. Consequently, the levels of measured biomarkers in oral rinses may be lower that other methods, prior to analytical processes.

Methods that collect specific molecules over extended time, such as 30 seconds, may be more reliable and obtain more detectable molecules than test strips and swabs. Whole-mouth rinses can release molecules trapped in biofilm, or in sublingual or gingival locations, and can access molecules located in certain mouth areas, such as the antierior tongue or buccal regions. Chew-stimulated saliva production may increase levels of proteases and other biomarkers (Al Habobe, H. et al., 2024, 1).

Among humans, whole mouth rinses have been shown to be superior to test strips or swabs or pads for collecting samples of periodontal biomarkers in patients. It was unexpectedly demonstrated that a simple mouth rinse with distilled water yields higher sensitivity to specific biomarkers than localized gingival sampling using swabs or sticks. This seems counter-intuitive, since the biomarkers of interest (matrix metalloproteinases (MMPs)) originate in gingival crevicular fluid, but whole-mouth rinse fluid provides greater biomarker yield, presumably by liberating molecules from subgingival locations throughout the mouth (Lähteenmäki, H. et al., 2019, 2).

Chew-stimulated saliva production has been found to increase salivary levels of certain proteins can be explained by the effect of mechanical stimulation. The parotid glands are strongly affected by mechanical stimuli, leading to an increase in salivary secretion. In particular, chew-stimulation increases levels of salivary amylases, which form complexes with salivary proteins, particularly proteases like MMPs (Dawes & Kubieniec, 2004, 3).

However saliva expectoration methods, both passive and chew-stimulated, and whole-mouth rinses, do not capture, collect, concentrate, and make available target molecules for diagnostic analysis, as do test strips and swabs that can be tightly configured with analytical systems.

Thus there is a need to provide systems that sample whole-mouth saliva, particularly including chew stimulated saliva production, including fluid to release specific molecules related to medical information from the entire mouth, and capturing, collecting, and concentrating the specific molecules for release on command for diagnostic analysis.

Whole-mouth saliva tests to diagnose periodontitis and other diseases in companion animals and domestic animals cannot use saliva expectoration methods and whole-mouth rinses, which require that patient's follow complex instructions. However test strips and swabs used for diagnosis are poorly tolerated in companion animals and domestic animals. This is why veterinarians put animals under complete anesthesia prior to oral examinations, which greatly limits veterinary dentistry. In dogs, a principal saliva collection method is found in at-home DNA collection kits that use foam sticks or cotton swabs to contact gingival margins, which (a) provide limited sample volume, (b) do not access whole mouth fluid, and (c) are poorly tolerated by animals.

There is thus a need for a non-invasive, animal-friendly, high-yield sampling method with the sensitivity of the human mouth-rinse technique, to capture, collect, and concentrate specific molecules in the sample related to veterinary information and release them on command for diagnostic analysis.

### SUMMARY OF THE INVENTION

The Hydrogel Ball includes a housing with a hydrogel inside. The hydrogel can be modified to bind biomarkers in saliva and release them on command. In preferred embodiments, the Hydrogel Ball is configured for use in the oral cavity of an animal to capture, collect, concentrate, and release salivary biomarkers. Although the oral cavity is a preferred location, the Hydrogel Ball may be adapted for use in other fluid-containing cavities of the body.

The housing comprises a flexible ball constructed from biocompatible materials suitable for oral contact. In preferred embodiments, the housing contains multiple holes that allow liquid to traverse freely between the interior and exterior of the device. In embodiments the housing is made from a flexible elastomer (silicone, TPE, medical-grade polyurethane, rubber), with holes that are perforations or pores (0.2-10 mm) sized to prevent the hydrogel materials from escaping while allowing efficient fluid exchange. In further embodiments, the housing contains nonwoven or woven meshes, or paper-like filters, that contain but let fluid reach the superabsorbent hydrogel. Embodiments use hydrogentangled nonwovens, alginate or chitosan felts, medical-grade PET or nylon mesh and ePTFE membranes, and silicone fabrics/spacer meshes. Embodiments use 3D spacer fabrics that lets the superabsorbent hydrogel swell without bursting seams.

As used herein, the term “hole” is intended to broadly encompass any opening in a material, regardless of scale or formation mechanism. The term includes, without limitation, apertures, which are relatively large, defined openings (for example, openings in meshes, screens, fabrics, or machined structures), and pores, which are relatively small openings (for example, micro- or nano-scale openings occurring naturally or formed during polymerization, foaming, sintering, or other processes). Accordingly, unless otherwise specified, references to “holes” in the present disclosure are meant to cover both macroscopic apertures and microscopic or nanoscopic pores within a material.

The housing is spherical, spheroidal, or cuboid, and preferably is 0.5-5 cm in diameter, the size depending on the oral cavity of the animal in which it is placed. There may be bumps or spike or fin-shaped appendages on the housing surface, to encourage mastication and prevent swallowing. The following Table 1 is a list of domestic and companion animal approximate Hydrogel Ball sizes for use in oral cavities, not including surface appendages.

TABLE 1

| Animal | Hydrogel Ball Diameter |
|---|---|
| Dogs (Canis familiaris) | |
| Small breeds (2-25 lbs) | 15-40 mm (0.6-1.6 inches) |
| Medium breeds (25-60 lbs) | 25-55 mm (1-2.2 inches) |
| Large breeds (60-90 lbs) | 40-75 mm (1.6-3.0 inches) |
| Giant breeds (90+ lbs) | 55-95 mm (2.2-3.75 inches) |
| Cats (Felis catus) | |
| All breeds | 12-30 mm (0.5-1.2 inches) |
| Horses (Equus caballus) | |
| Adult | 80-155 mm (3.1-6 inches) |
| Ponies | 60-100 mm (2.4-3.9 inches) |
| Draft | 100-160 mm (3.9-6.3 inches) |
| Goats (Capra aegagrus) | |
| Dwarf/Pygmy | 25-60 mm (1.0-2.4 inches) |
| Sheep (Ovis aries) | 35-70 mm (1.4-2.75 inches) |
| Humans (Homo sapiens) | |
| Adult | 25-50 mm (1.0-2.0 inches) |
| Ages 13-17 | 25-35 mm (1.0-1.4 inches) |
| Ages 6-12 | 20-30 mm (0.8-1.2 inches) |
| Ages 2-5 | 15-25 mm (0.6-1.0 inches) |

The interior of the Hydrogel Ball contains superabsorbent hydrogels based on biocompatible carbohydrate-type polymers. These can be composed of alginate, cellulose, starch, chitosan, crosslinked polyacrylate blends, sodium polyacrylate, PEG-modified polysaccharides, and other food-grade or medical-grade superabsorbent materials. The hydrogels are capable of holding 10-1000 times their dry weight in water. They may be optionally crosslinked with calcium or borate for structural integrity.

The hydrogels are modified to incorporate specific binding agents to capture, collect, and concentrate specific diagnostic biomakers found in saliva. Saliva has a complex composition that includes salivary and gingival biomarkers that can be measured for the early diagnosis of some oral and systemic diseases, including periodontitis, diabetes mellitus, pancreatic cancer, atopic dermatitis, residual leukaemia disease, chronic kidney disease, and prostate cancer. Hydrogel binding agents can be covalently attached to the hydrogel matrix or physically entrapped within the polymer network. Following capture and concentration of target enzymes, they are released from the hydrogel for analysis.

Hydrogel Ball embodiments may incorporate monoclonal or polyclonal antibodies in the interior hydrogel, that bind biomarkers that enter the hydrogel. Antibody generation is a well understood art. In the traditional method, the target protein or fragment is injected into an animal, and the antibodies the animal makes in response are collected. For monoclonal antibodies, one antibody-producing cell is isolated, the fused with an immortal cell to make a hydrodoma from which copies are generated. For polyclonal antibodies, a mix of antibodies to many parts of the protein are collected from the animal's blood serum. Recent advances in antibody generation do not use animals and may be more specific. Recombinant antibodies are built from amino acids in a lab. Phage display uses a library of billions of random antibody fragments attached to phage viruses; a protein introduced to the library isolates specific fragments that target the protein. Synthetic binding molecules are generated in silico, on a computer, often making aptamers that behave like antibodies, but are much shorter. All of these methods may be used to generate antibodies and other fragments that are attached to hydrogel matrices, generally on short flexible tethers, where the antibodies and other fragments can bind biomarkers that enter the hydrogel.

Hydrogel Ball embodiments capture, collect, and concentrate biomarkers with binding agents incorporated in the hydrogel that may include hydroxamate groups targeting zinc-binding metalloproteinases, collagen-mimetic triple helices, bivalent bait+inhibitor constructs, TIMP-mimetic peptides, antibodies, aptamers, heparin or sulfated glycosaminoglycans, ion-exchange groups, and nucleic acid hybridization probes (DNA, Locked Nucleic Acid (LNA), and Peptide Nucleic Acid (PNA)). Biomolecules such as MMPs, microRNAs, and metabolites including urea are enriched within the hydrogel during contact with saliva or another fluid. These binding agents are displayed on short PEG/Gly/Ahx linkers at moderate density and grafted by standard chemistries (e.g., 1-ethyl-3-(3-dimethylaminopropyl) carbodiimide (EDC)/N-hydroxysuccinimide (NHS), maleimide-thiol, (desthi)biotin-streptavidin).

After biomarkers are captured and collected, a Hydrogel Ball releases them with a variety of embodiments: programmed triggers include reducing agents, light activation, enzyme-cleavable linkers, pH or ionic strength changes, competitive ligands, or strand-displacement oligonucleotides. Vesicle-centric workflows add permeabilization (saponin/digitonin, local salt/pH microdomains, freeze-thaw, nanoparticle triggers) to expose intravesicular RNA before hybrid capture. The hydrogels enable collection, concentration, and on-demand release of biomarkers suitable for downstream analysis.

The following are disease and conditions that can be detected with salivary biomarkers:

TABLE 2

| | | |
|---|---|---|
| Oral and Dental | Systemic Cancers | Cardiovascular Disease |
| Diseases | Breast cancer | Myocardial infarction |
| Periodontal disease | Pancreatic cancer | Coronary artery disease |
| Dental caries | Lung cancer | Heart failure |
| Oral cancers | Gastric cancer | |
| Sjögren's syndrome | Ovarian cancer | |
| Metabolic and | Infectious Diseases | Neurological/Psychiatric |
| Endocrine Disorders | HIV/AIDS | Conditions |
| Diabetes (Type 1 and | Hepatitis viruses | Alzheimer's disease |
| Type 2) | (A, B, C) | Parkinson's disease |
| Metabolic syndrome | SARS-CoV-2 | Depression |
| Thyroid disorders | Herpes viruses | Schizophrenia |
| | Measles | Autism spectrum |
| | Mumps | disorders |
| Autoimmune Diseases | Other Conditions | |
| Sjögren's syndrome | Kidney disease | Drug exposure and |
| Systemic lupus | Liver disease | monitoring |
| erythematosus | Stress/cortisol-related | |
| Rheumatoid arthritis | conditions | |

The following describes Hydrogel Ball embodiments that capture, collect, concentrate, and release on command, saliva biomarkers used to diagnose three representative diseases of humans and other animals: periodontitis, pancreatic cancer, and chronic kidney disease. Also Hydrogel Ball embodiments are described that help determine levels of a psychological state, stress. In each of these examples, what is described are systems and methods incorporating molecules in the Hydrogel Ball hydrogels, their binding action, and release on command processes. These are representative examples of the system and methods that can incorporate binding agents in Hydrogel Ball hydrogels that capture, collect, concentrate, and release on command medical and veterinary biomarkers for diagnosis. The hydrogels are included within Hydrogel Balls that people and other animals may hold in their mouths, which expel and absorb fluid when their mouths “chew” or “squeeze” them, the fluid flow bringing salivary compounds into the hydrogel.

### Periodontitis

MMPs, particularly MMP-8 and MMP-9, are established biomarkers of periodontal tissue breakdown. In a preferred embodiment, Hydrogel Ball hydrogels are modified to incorporate specific binding agents to enhance MMP capture and concentration. These binding agents can be covalently attached to the hydrogel matrix or physically entrapped within the polymer network. Following capture and concentration of target enzymes, they are released from the hydrogel for analysis.

MMP collagenases like MMP-8 are enzymes that cut collagen and related peptides. Normally when they enter a hydrogel they grab and cut peptides, one after the other. To trap MMPs in the Hydrogel Ball hydrogel they must be stopped from cutting. That requires a way to hold them fast. Later, the bound, collected MMPs must be released for analysis.

One embodiment (“Embodiment A”) binds MMPs by exploiting the zinc atoms that MMPs use to cuts collagen. Hydroxamate is very good at attaching to zinc, and is incorporated in the Hydrogel Ball hydrogel at the end of flexible PEG chains. When MMPs enter the Hydrogel Ball hydrogel, their zinc atoms attract to, and attach to, one the many hydroxamate molecules around. The enzyme is then stuck—it can't continue cutting.

The PEG tethers that binds hydroxamate can be broken on command. One way to form a cleavable PEG tether is to make the PEG-hydroxamate hydrogel with a disulfide bond. After MMPs become attached, adding a reducing agent (such as glutathione, DTT, or TCEP) breaks the disulfide and frees the hydroxamate-enzyme complex from the gel. Following this the hydroxamate, and the MMP enzyme stuck to it, detach from the hydrogel, float into solution, and can be collected and analyzed.

Another embodiment (“Embodiment B”) holds fast MMPs with a Hydrogel Ball hydrogel that includes many synthetic peptides that behave like tiny collagen pieces. MMP collagenases are attracted to these peptides, which resemble the collagen the enzymes cut. Real collagen is a triple helix twisted together. Synthetic versions called triple-helical peptides can be generated with the same shape. MMP collagen-cutting enzymes have a central structure that fits on triple helices. When the enzymes bump into synthetic versions, they bind to the groove in it that, in real collagen, is also where they cut through it. But the synthetic versions, despite having similar structure, cannot be cut. Instead the MMP is stuck.

To release the MMPs bound to synthetic peptides, several methods can be used. In one embodiment soluble collagen-like pieces in a solution surrounding the gel induce MMPs to release from their synthetic peptides and enter the solution. In a second embodiment a zinc chelator is added to the Hydrogel Ball hydrogel, which weakens MMP attachments. The hydrogel is rinsed and enzymes enter solution. In a third embodiment temperature or salt levels are increased, which melts the triple helices, releasing bound MMPs. In a fourth embodiment the PEG or glycine-rich linkers that tie the triple helices to the hydrogel are dissolved by reducing agents or enzymes.

In another embodiment (Embodiment “C”), the Hydrogel Ball hydrogel incorporates a bivalent capture system comprising a collagen-mimetic peptide and a zinc-binding hydroxamate group linked by a cleavable, flexible spacer. The peptide engages the enzyme's substrate-binding groove while the hydroxamate chelates its catalytic zinc, forming a dual-point, high-affinity complex. Suitable spacers include short polyethylene glycol chains, glycine repeats, or aminohexanoic acid linkers of approximately 2-3 nm in length, sufficient to maintain dual binding while limiting excessive flexibility.

After MMP are collected, they are later released. One embodiment incorporates disulfide bonds that break when exposed to mild reducing agents (like glutathione). Another embodiment incorporates potoclavable bonds that dissolve when exposed to certain light wavelengths (e.g., violet light). Another embodiment incorporates short enzyme-cleavable sequences that cannot be cut by MMPs, but can be cut by another, harmless enzyme. When this enzyme is added, MMPs are released.

Another embodiment (Embodiment “D”) uses a mimic of a protein called Tissue Inhibitor of Metalloproteinase, or TIMP. In the body, TIMPs cover proteases such as MMPs and prevent them from cutting too much collagen. By synthesizing just part of a TIMP, its critical, short peptide loop that grabs onto MMPs, and anchoring these peptides to a Hydrogel Ball hydrogel, MMPs that bump into them are bound and held tightly. TIMPs are more specific than bait peptides, because TIMPs evolved to bind MMPs. TIMP mimics are also releasable with a chemical wash, light, or another enzyme.

In another embodiment (Embodiment “E”), the Hydrogel Ball hydrogel incorporates heparin or sulfated glycosaminoglycan chains that electrostatically bind proteases such as MMPs. Heparin may be co-polymerized with hydrogel precursors, introduced as heparin-methacrylate or heparin-maleimide, or grafted post-polymerization through carbodiimide coupling between carboxyl and amine groups. Bound enzymes are released by high-salt or competitive heparin solutions.

In another embodiment (Embodiment “F”), the Hydrogel Ball hydrogel presents antibodies tethered by short flexible linkers to permit exposure of antigen-binding regions. Orientation may be achieved through Protein A/G scaffolds that bind the Fc region of IgG, or by site-specific Fc modification using click chemistry, leaving Fab regions accessible for target interaction.

In an embodiment the Hydrogel Ball hydrogel can be rinsed with a mildly acidic buffer or other pH shift to release collected MMPs. In another embodiment adding a short peptide from the MMP's binding site to solution will induce MMPs to detach from antibodies. In another embodiment addition of salt or very mild chaotropes can weaken binding. In other embodiments the antibodies are attached to the hydrogel with cleavable links, and MMPs are released via chemical wash, light, or another enzyme.

Another embodiment (Embodiment “G”) uses short DNA/RNA strands (aptamers) or small peptides that are lab-evolved to fit tightly onto a specific protein, like an MMP. Distributing these aptamers or peptides in a Hydrogel Ball hydrogel catches proteins that enter—the aptamer folds around it, or the peptide latches onto a patch of the enzyme's surface. The aptamers and peptides are non-covalent, so they release the protein when the environment changes or with a trigger.

In another embodiment (Embodiment “H”), the Hydrogel Ball hydrogel incorporates ion-exchange groups that attract proteins according to surface charge. Cation-exchange groups capture proteins with positive domains, and anion-exchange groups capture proteins with negative domains at the working pH. Electrostatic binding is reversible and may be released by adjusting salt concentration or pH. Although non-specific, ion exchange can serve as a preliminary enrichment step before specific analysis. In certain embodiments, released MMPs are stabilized in a physiological buffer containing Zn²⁺, Ca²⁺, and neutral pH.

The various embodiments for capturing MMPs and releasing them can be combined in various ways. Ion-exchange hydrogels that pre-concentrate many proteins can be combined with heparin that enriches proteases like MMPs, and aptamers/antibodies that specifically capture the MMP of interest.

The various embodiments are made reversible in several ways, which can also be combined. Disulfide bonds can be broken with TCEP/DTT; photocleavable bonds can be severed with specific light wavelengths; free heparin, soluble peptide, inhibitor, or antigenic peptide can compete with the binding force on MMPs, releasing them; and ionic/pH switches that can dissolve bonds.

pH sensitive switches are molecules that break apart when the environment becomes more acidic or basic, releasing whatever is attached to them. An embodiment uses alginate or polyethylene glycol as hydrogel material, with aldehyde groups attached to some of the polymer chains. Hydrazone groups are bound the aldehyde. Hydrazone is generally stable at pH˜7.4 but dissolves at pH below ˜6.5. An embodiment uses acetal/ketal bonds, also acid-sensitive.

Another embodiment adds EDTA in the hydrogel precursor, or covalently bonds EDTA to gelled hydrogel, to form a latent MMP release agent. EDTA grabs zinc from MMPs, pulling the zinc ions out of their molecular pocket. This reduces MMP binding affinity. Under normal conditions EDTA will remain inactive in the hydrogel because the MMP zinc is protected by proteins. Then lowering pH activates the EDTA and causes enzyme release. In another embodiment compression or reducing agents cause the EDTA to dissolve MMP bonds. In another embodiment EDTA is loaded into hydrogels in a delayed release form, encapsulated in microspheres that degrade gradually. As the EDTA is released, it pulls MMP zinc ions out of their pocket, and reduces MMP binding affinity. In an embodiment EDTA binds MMPs and zinc buffer restores MMP activity.

Another embodiment uses an auto-catalytic tether system that incorporates a cleavage sequence as well as a binding domain that captures MMP. Certain exact amino acid sequences, such as Pro-Gln-Gly-Leu-Ala-Gly, can be cut by specific MMPs. MMPs recognize these sequences, independently of the zinc ions. A binding system is needed to hold the MMP against diffusion. To release the MMPs, the chelating agents are washed out and the binding agent is dissolved. This can involve competing chelators, pH changes that weaken chelation, high salt or excess zinc.

Many other salivary biomarkers have been studied in relation to periodontitis, including inflammatory markers (CRP and calprotectin), cell activity markers (alkaline phosphatase, lactate dehydrogenase, aspartate aminotransferase, and alanine aminotransferase), and growth factors (hepatocyte growth factor). Interleukin-17, Interleukin-23, and Interleukin-1 beta are pro-inflammatory cytokines that contribute to periodontal tissue damage and disease severity. These can be captured, collected, and concentrated in Hydrogel Balls.

### Pancreatic Cancer

MicroRNAs are very short, non-coding RNA molecules (˜20-24 nucleotides long) that regulate DNA processess. Saliva levels of certain microRNAs, such as hsa-miR-21, hsa-miR-23a, hsa-miR-23b, miR-29c, hsa-miR-216, miR-3679-5p, and miR-940, are significantly increased (or decreased) in patients with pancreatic cancer. Specifically miR-3679-5p and miR-940 have an excellent ability to detect very early-stage pancreatic cancer, which enables curative surgery. Under normal circumstances the disease has a 95% mortality rate. Note that in saliva, microRNAs are often encapsulated in exosomes (vesicles) or bound to proteins (like Argonaute), to protect delicate RNA from enzymes (Xie, Z. et al., 2015, 4).

In an embodiment (Embodiment “I”) a Hydrogel Ball hydrogel contains many PEG chains with DNA, LNA, or PNA at their ends. The DNA-like sequences are used because they are stronger, more selective, and resistant to enzymes than real DNA. When a particular microRNA approaches, DNA or DNA-like sequences that form a complement to the particular microRNA hybridize with it (form base pairs).

Another embodiment (Embodiment “J”) exploits RNA's negative charge to bind microRNAs. Short regions of chitosan, poly-L-lysine, or cationic PEG are added to the Hydrogel Ball hydrogel, producing localized positive charges in the hydrogel. RNA and RNA-carrying vesicles preferentially attach to these areas, concentrating total RNA and RNA-carrying vesicles in the hydrogel.

In another embodiment (Embodiment “K”) the Hydrogel Ball hydrogel is functionalized with antibodies or affinity ligands that recognize exosome surface markers. Tetraspanins such as CD63, CD9, and CD81, ESCRT-associated proteins, integrins, ICAM-1, lamp proteins, or heat shock proteins may be targeted. The antibodies are attached to the hydrogel via chemistries such as EDC/NHS or click-chemistry, and optional engineered spacers include disulfide or enzyme-cleavable linkages to enable on-demand release of intact vesicles. Exosome release may also be triggered by pH shift, addition of excess ligand peptides, neutralizing antibody fragments, or salt/chaotrope exposure. In other embodiments, exosomes captured in the hydrogel are permeabilized in situ by release of detergents (saponin, digitonin), exposure to localized salt or pH stress, or thermal cycling, thereby rendering their internal microRNA cargo accessible for probe hybridization.

### Chronic Kidney Disease

Studies find a statistically significant positive correlation between elevated levels of salivary urea and clinical stages of chronic kidney disease (Lasisi, T. J. et al., 2016, 5). Although both salivary creatinine and urea have good sensitivity and specificity values, of these and other organic compounds, salivary urea levels correlate with different levels of kidney function. (Poposki, B. et al., 2023, 6).

A Hydrogel Ball embodiment (Embodiment “L”) incorporates hydrogel that passively captures salivary urea in urea-shaped micro-cavities in the hydrogel matrix. A hydrogel precursor is mixed with a little urea that is bound to monomers. After the hydrogel cures, the urea in it is washed out, leaving the imprinted urea sites behind. When saliva with urea enters the hydrogel, the urea fits the tiny cavity sites and concentrates in the hydrogel. To release the captured urea, the hydrogel is rinsed with water or mild buffer, which washes urea from the capture sites into sample fluid that serves as an extract for a standard urea test.

An embodiment (Embodiment “M”) makes a Hydrogel Ball hydrogel with soft precursors, and adds negatively charged groups (such as sulfonate or carboxylate) and a small amount of urease. Saliva diffuses in and urease converts urea to ammonia and carbon dioxide. The negatively charged groups bind the positively charged ammonium, concentrating the urea signal as captured ammonium. The ammonium is eluted with a small salt or mild base rinse, and urea can be measured indirectly (via ammonia).

### Stress and Cortisol-Related Conditions

Salivary cortisol is an excellent marker for acute stress responses but shows complex, sometimes paradoxical patterns in chronic stress and anxiety disorders. Context, timing, and individual factors are crucial. Hydrogel Ball embodiments for athletes enable detection of incomplete recovery from training and anxiety and readiness states. Embodiments for test anxiety identify students with excessive anxiety that may impair performance. Embodiments for decision-makers identify when stress levels may compromise decision-making. Embodiments can be used in therapy, to track physiological habituation. Embodiments for pilots, surgeons, and emergency responders may help these people understand their stress baselines before critical tasks. These embodiments may be personal information that an individual interprets given their own histories. It may be more important to track the pattern of cortisol levels over days and weeks, that use a single value.

A Hydrogel Ball embodiment (Embodiment “N”) uses a molecularly imprinted hydrogel with cortisol-shaped cavities to bind salivary cortisol. A user masticates the Hydrogel Ball for predetermined period, then removes the Hydrogel Ball, opens the housing, and inverts the hydrogel into a device container. In the device the hydrogel is rinsed then bound cortisol is elated. The user puts a certain volume of the eluate on a standard cortisol test strip. After a stated time, the user reads a colored line that determines cortisol level. In another embodiment, the eluate is mixed with a cortisol-binding DNA aptamer that shifts the eluate color, visible by eye or phone camera. The color is compared to standard color curve to determine cortisol volume. An embodiment uses Enzyme-Linked Immunosorbent Assay (ELISA) to determine cortisol levels in solution. Cortisol in the sample competes with enzyme-labeled cortisol for antibody binding. An embodiment uses Chemiluminescent or Fluorescent Immunoassay. This works like ELISA but uses a light-emitting tag instead of a color change, improving sensitivity.

An embodiment immobilizes anti-cortisol IgG/Fab within a Hydrogel Ball hydrogel using Fc-selective covalent attachment to maintain exposed binding sites; the hydrogel is regenerable through competitive or mild solvent elution followed by neutralization and re-equilibration. Another embodiment incorporates corticosteroid-binding globulin (CBG) as a receptor-decoy for high-affinity cortisol capture, likewise regenerable under mild elution. A further embodiment grafts β-cyclodextrin or forms hydrophobic domains in the hydrogel, providing inclusion or partition sites that selectively bind cortisol.

The Hydrogel Balls disclosed provide modular, reversible, and multiplexed capture of diverse biomarker classes from saliva or other fluids. They enable in situ enrichment of low-abundance targets, protection of fragile analytes during sampling, and controlled release for analysis. These systems are compatible with diagnostic workflows for periodontal disease, cancer, kidney disease, and other conditions where salivary biomarkers are clinically relevant.

While the invention has been described in connection with certain exemplary embodiments, it is to be understood that the invention is not limited to those embodiments. On the contrary, various modifications, equivalents, and alternatives will be apparent to those skilled in the art without departing from the spirit and scope of the invention as defined by the appended claims.

### BRIEF DESCRIPTION OF THE DRAWINGS

FIGS. 1A and 1B are cross-sectional views of a Hydrogel Ball.

FIGS. 2A and 2B are cross-sectional views of a Hydrogel Ball.

FIG. 3 is a perspective view of one embodiment of a Hydrogel Ball.

FIG. 4 is a flow-chart of how a Hydrogel Ball is used.

FIG. 5 is a flow-chart of making a hydrogel that traps MMPs.

FIG. 6 is a flow-chart of making a dual-mode MMP capture system.

FIG. 7 illustrates a Hydrogel Ball hydrogel being processed for eluate.

### DETAILED DESCRIPTION OF THE INVENTION

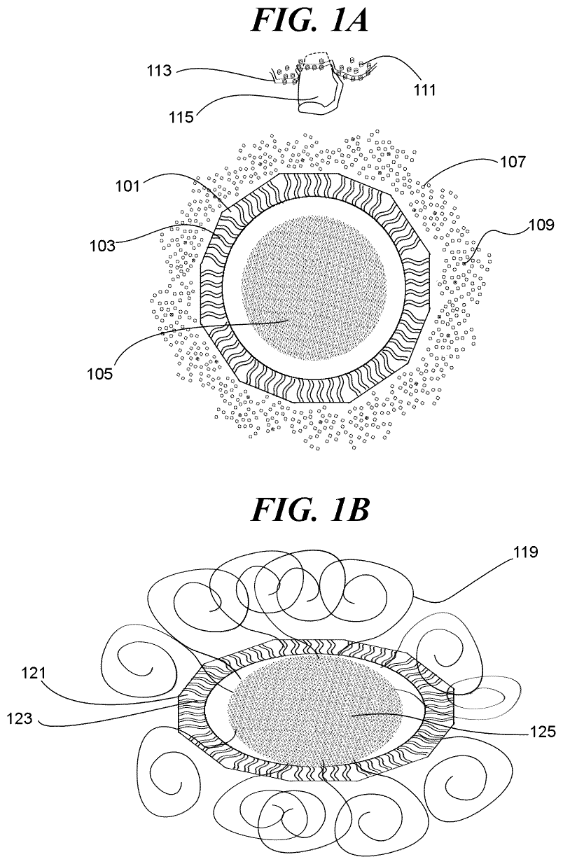

FIG. 1A illustrates a Hydrogel Ball in cross-section. A housing 101 is composed of biocompatible materials suitable for oral contact. The housing 101 is preferably 0.5-5 cm in diameter, the size depending on the oral cavity of the animal in which it is placed. In an embodiment holes 103 are perforations or pores 0.2-10 mm in diameter. In another embodiment the housing contains nonwoven or woven meshes, or paper-like filters, that contain but let fluid pass through, which contain holes 103 that may range from nanometer to millimeter diameter size. A hydrogel 105 that is superabsorbent, based on biocompatible carbohydrate-type polymers, is inside housing 101. The hydrogel 105 incorporates specific binding agents to capture, collect, and concentrate specific diagnostic biomakers 109 found in saliva 107. Biomarkers of interest 111 are also found in gingival margins 113 around a tooth 115.

FIG. 1B illustrates a Hydrogel Ball in cross-section, as it is compressed in an oral cavity. Housing 121, being composed of flexible elastomers, is compressed or elongated by animal mastication. The appropriately hydrated hydrogel 125 releases fluid 119 into the oral cavity through holes 123 when the housing 121 is compressed or elongated.

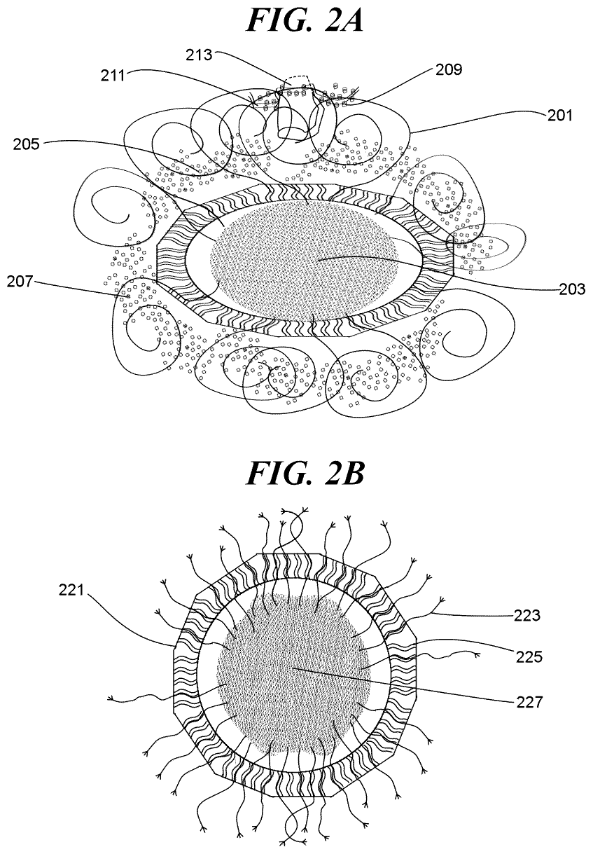

FIG. 2A illustrates a Hydrogel Ball in cross-section, during compression in an oral cavity. The hydrogel 203 releases fluid 201 into the oral cavity. The released fluid 201 mixes with saliva 205 from throughout the mouth, including specific diagnostic biomarkers 207 found in saliva 205. The released fluid 201 mixes with biomarkers of interest 209 found in gingival margins 211 around a tooth 213.

FIG. 2B illustrates a Hydrogel Ball in cross-section, after compression in an oral cavity. The housing 221 expands to return to its resting state, which causes the hydrogel 227 to expand and thereby reabsorb a water-saliva mixture 223 from the oral cavity through holes 225. The water-saliva mixture 223 contains specific diagnostic biomarkers and biomarkers of interest that interact with binding agents in the hydrogel 227. When the Hydrogel Ball is removed from the animal's mouth after a predetermined or device determined period, the hydrogel now contains reabsorbed oral fluid with biomarkers.

The hydrogel is processed to recover the concentrated sample. If binding agents used, altered equilibrium conditions cause release of bound biomarkers.

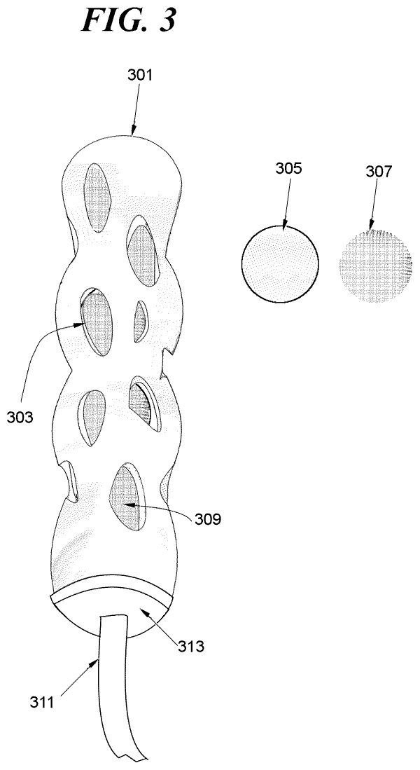

FIG. 3 illustrates a tethered chew-stick embodiment. A soft housing 301 surrounds made of a silicone material encapsulates multiple hydrogel spheres 305, each enclosed in a fine mesh 307 that contains nano or micro-sized holes for fluid to transit. A combination 309 of hydrogel 305 and fine mesh 307 are positioned in chambers 303 along the length of the soft housing 301. At one end of the soft housing is a tether 311 attached to a removable structure 313 that is connected to the combinations 309, and the combinations 309 can be removed with the removable structure 313.

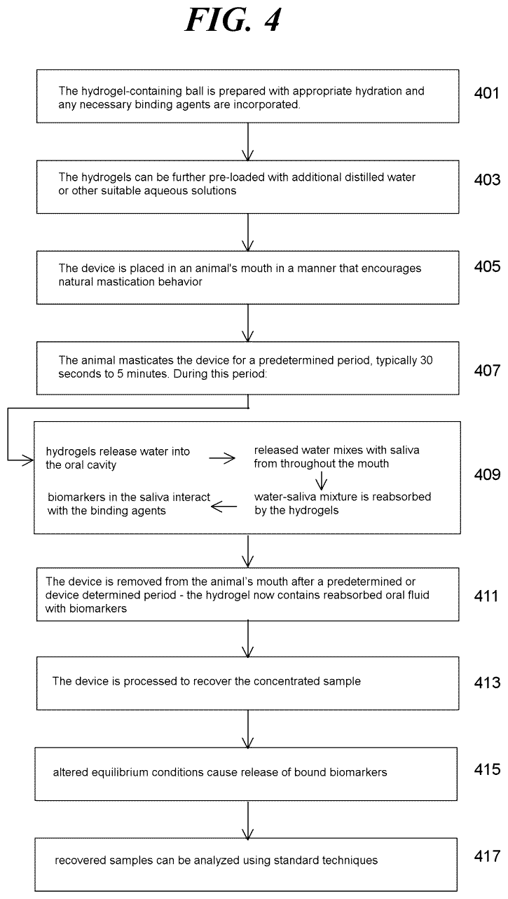

FIG. 4 is a flow chart of a method of use of the Hydrogel Ball. Prior to use the Hyrogel Ball is prepared with appropriate hydration and any necessary binding agents are incorporated 401. In preparation for sampling, the hydrogels can be further pre-loaded with additional distilled water or other suitable aqueous solutions (0.5-3 mL) 403. For use of the Hydrogel Ball, the device is placed in an animal's mouth in a manner that encourages natural mastication behavior 405. A mastication period occurs, as the animal masticates the device for a predetermined period, typically 30 seconds to 5 minutes 407. During this period 409, the hydrogels release fluid into the oral cavity, the released fluid mixes with saliva from throughout the mouth, then the water-saliva mixture is reabsorbed by the hydrogels, then biomarkers in the saliva interact with the binding agents in the hydrogels. The Hydrogel Ball is removed 411 from the animal's mouth after a predetermined or device determined mastication period. The hydrogel now contains reabsorbed oral fluid with biomarkers. The device is processed to recover the concentrated sample 413. If binding agents used, altered equilibrium conditions cause release of bound biomarkers 415. Some of these altered conditions include mechanical compression (syringe plunger or press), centrifugation in containment tube, or chemical release (buffered saline, pH shift) eluting biomarkers.

The recovered samples can be analyzed using standard techniques 417. In embodiments for MMP detection and quantification, these include enzyme-linked immunosorbent assays (ELISA), lateral flow assays (LFA), fluorometric assays (FA) and the many specialized FA types, zymography, mass spectrometry, western blotting, as well as commercial methods.

MMPs use a catalytic Zn²⁺ to cut collagen. Hydroxamate groups bind Zn²⁺ with high affinity. An embodiment presents hydroxamates at the ends of flexible PEG chains throughout the gel. When an MMP diffuses in, its Zn²⁺ is chelated and the enzyme is effectively parked (catalysis halted). The MMP is released by building a cleavable link into the tether (a disulfide). A mild reducing agent (glutathione, DTT, or TCEP) breaks the disulfide, releasing the hydroxamate-MMP complex into solution for collection and analysis.

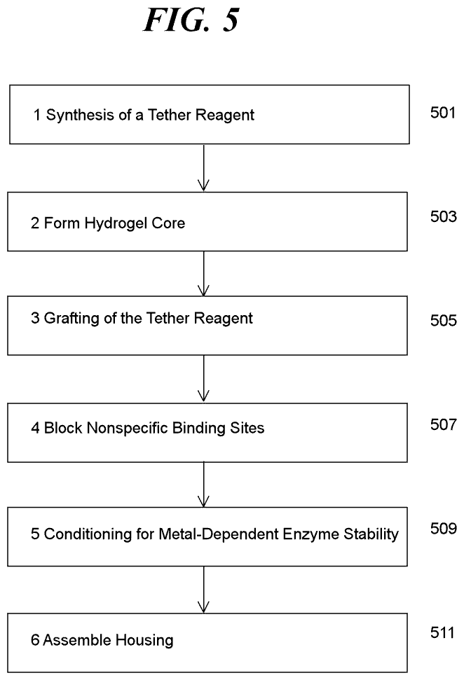

FIG. 5 outlines an embodiment (Embodiment A) that makes a hydrogel that traps MMPs via Zn²⁺ chelation and releases them on-demand through reductive cleavage, composed of six steps. Each of the six steps is detailed below

Step 1: Synthesis of a Tether Reagent 501 In certain embodiments, a tether molecule is prepared that comprises:
(1) a metal-chelating moiety capable of binding zinc ions (for example, a hydroxamate, catechol, or carboxylate group);
(2) a flexible hydrophilic spacer (for example, a polyethylene glycol chain);
(3) a cleavable linker (for example, a disulfide or other redox- or photo-cleavable group); and
(4) a reactive terminal group (for example, a maleimide, vinylsulfone, or acrylate) configured to couple to thiol or amine groups within a hydrogel network.

In one illustrative synthesis, a PEG chain containing an N-hydroxysuccinimide (NHS) ester and a thiol-reactive group is reacted with a hydroxamate or related zinc-binding molecule in an anhydrous solvent with a tertiary amine catalyst to yield a hydroxamate-PEG-disulfide-maleimide intermediate. he product may be purified by precipitation, dialysis, or chromatography and confirmed by spectroscopic or mass-spectrometric analysis. Functionally equivalent linkers, reactive termini, and coupling chemistries are contemplated.

### Step 2: Form Hydrogel Core 503

A soft, thiol-containing hydrogel matrix is prepared from hydrophilic monomers or polymers bearing reactive groups such as thiols, acrylates, or vinylsulfones. Exemplary materials include multi-arm PEG-thiol, PEG-diacrylate, gelatin-SH, alginate-SH, or similar precursors. Crosslinking is achieved by photoinitiation, thermal initiation, or redox initiation under aqueous conditions to form a lightly crosslinked network with residual reactive sites available for post-functionalization. Hydrogel stiffness and porosity are adjusted by varying polymer concentration or crosslink density to achieve a matrix capable of absorbing biological fluids while allowing enzyme diffusion.

### Step 3: Grafting of the Tether Reagent 505

The preformed hydrogel is contacted with the tether reagent under mild aqueous conditions to enable covalent coupling through the thiol-reactive group (for example, thiol-maleimide or thiol-Michael addition chemistry). This step introduces the metal-chelating groups on flexible linkers throughout the hydrogel. Unreacted reagent is removed by repeated washing in buffer until absorbance or conductivity stabilizes. Equivalent coupling chemistries such as amine-NHS condensation, azide-alkyne click, or carbene insertion may also be used to attach the tether to the hydrogel.

### Step 4: Block Nonspecific Binding Sites 507

After tethering, nonspecific adsorption sites are minimized by exposure to a blocking or passivation solution. Such solutions may contain inert proteins (e.g., bovine serum albumin, casein), polysorbates, or polyethylene glycol derivatives. Blocking reduces nonspecific protein binding and preserves selectivity of the metal-binding domains.

### Step 5: Conditioning for Metal-Dependent Enzyme Stability 509

Prior to use, the hydrogel is equilibrated in a buffer that maintains activity of zinc-dependent proteases. The buffer typically contains a buffering agent (for example, HEPES, Tris, or phosphate), salts providing calcium and zinc ions, and optional stabilizers or surfactants to maintain enzyme solubility. The hydrogel may be stored in this buffer under refrigeration or frozen with cryoprotectants for long-term use.

This conditioning ensures that when the hydrogel contacts enzyme-containing biological fluids, active enzymes are chelated by the tether's metal-binding groups and retained within the hydrogel.

### Step 6: Assemble Housing 511

The functionalized hydrogel is enclosed within a housing configured to permit fluid exchange while retaining the gel. The housing may be a perforated or porous structure formed from polymers such as silicone, polyurethane, PET, PLA, or thermoplastic elastomers, optionally fabricated by molding, machining, or additive manufacturing. Openings may range from sub-micrometer pores to millimeter-scale perforations to facilitate controlled diffusion.

The hydrogel is placed inside and secured by a mechanical closure, snap-fit, or adhesive seal, forming a self-contained Hydrogel Ball device suitable for placement in the oral cavity or other body environments for enzyme capture.

After use, the hydrogel can be removed and treated with a reducing agent (for example, dithiothreitol (DTT), tris(2-carboxyethyl) phosphine (TCEP), or glutathione) to cleave the disulfide or other reducible linker, thereby releasing the captured enzyme-tether complexes into solution for analysis.

Embodiment A is modular; each phase can be optimized independently. It is scalable; batch-produce gels can be assembled on demand. The housing is versatile; ball design can be adapted for different deployment scenarios The hydrogel has low nonspecific binding; BSA/casein blocking eliminates artifacts. Pre-conditioning prevents MMP denaturation. The sealed housing protects the hydrogel during handling. However elegant, however, this is one design among many possible.

Embodiment B captures enzymes like MMP by exploiting how they recognize and bind collagen's triple helix geometry. By presenting non-cleavable triple-helical peptides (THP) mimics, the enzyme docks into its binding groove but cannot complete catalysis, becoming kinetically trapped.

There are at least three ways to make THP mimics that are three amino-acid strands and non-cleavable. In a preferred embodiment, a scissile amide is replaced with thioamide. This single amino-acid change lets the enzyme bind but prevents it from hydrolizing the carbon to sulfur bond that holds the linkers together. Another embodiment changes the amino-acid L-glycine to d-glycine, it's mirror-image form. This blocks the enzyme's catalytic mechanism. Another embodiment replaces a peptide bond between two amino-acids with a polar bond with higher electronegativity, altering linker shape.

To synthesize THPs, solid-phase peptide synthesis is used, combining standard amino-acid coupling with a special dipeptide in the cleavage site. The critical process is to form single amino-acid strands into a trimer. This can be done by dissolving them in pH˜3 acid, concentrating this, heating it to 50° C., cooling to 4° C., neutralizing to pH 7, then incubating at 4° C. The resulting THPs can be grafted in a hydrogel with click chemistry, incorporating PEGDA for additional cross-linking, adding a copper catalyst for covalent links, plus a copper stabilizing ligand. After gel formation, the copper has to be washed out, because residual Cu²⁺ interferes with MMPs. The THP hydrogel density is ˜0.5-2 mM.

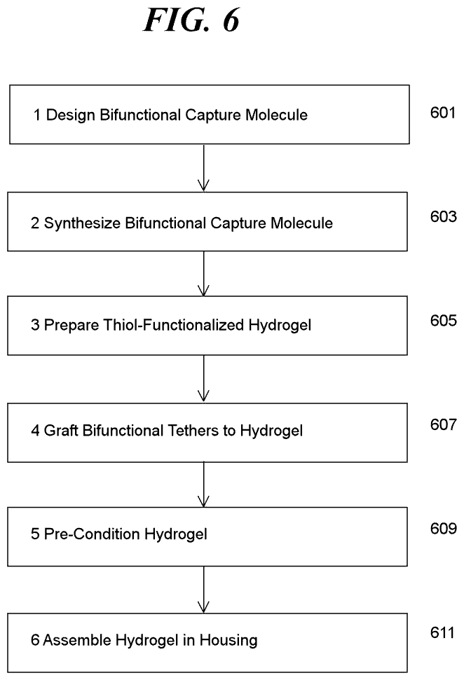

Embodiment C combines bifunctional peptide-hydroxamate tethers in a hydrogel, which can increase enzyme affinity, selectivity, and capture strength. This includes hydrogel trap that includes both a collagen-like bait and a zinc-binding inhibitor on a breakable tether. An MMP enzyme is pulled in and bound at two places, once in the groove where it does its cutting, and also at its zinc atom. That forms an especially strong, specific bond. Both the small collagen-mimic peptide (e.g. GPQGL↑WGQ) that attracts the enzyme, and the hydroxamate group that sticks to zinc, are linked with short flexible spacers, such as a short PEG chain (PEG2-PEG6), glycine repeats (a string of glycine residues such as Gly-Gly-Gly, etc.), and/or aminohexanoic acid (a six-carbon flexible spacer inserted between a peptide and a small molecule like a hydroxamate that is long enough to add wiggle room). The spacers are around 2 to 3 nm long, which allows the hydroxamate to reach the zinc while the peptide stays lodged in the enzyme's substrate groove, but short enough to keep the peptide and hydroxamate from excessive movement. FIG. 6 is a flow chart of the six key steps in generating a dual-mode MMP capture system. The six steps are explained below.

Step 1 designs a molecule with both substrate recognition and zinc chelation 601. It combines the maleimide—SS click handle with the cleavable peptide and a PEG4 spacer of 2-3 nm length and a hydroxamate Zn²⁺ chelator. Step 2 builds three modules, then joins them together 603. Module 1 uses solid-phase peptide synthesis to make a Mal-SS-Peptide. Module 2 forms a bifunctional compount, PEG4-ABHA, a spacer chain and an active chemical group that can be conjugated to other molecules. Module 3 couples the Mal-SS-Peptide with PEG4-ABHA. Step 3 creates a soft hydrogel with free pendant thiols for maleimide click 605. Step 4 click captures molecules onto hydrogel thiols, using thiol-maleimide click 607. Step 5 blocks nonspecific binding and equilibrates the hydrogel for MMP stability 609. The hydrogel is rinsed with a blocking buffer, then incubated with an MMP assay buffer. Step 6 assembles the hydrogel in the housing 611.

Embodiment D uses a TIMP-mimetic display to catch MMPs. TIMP proteins naturally inhibit MMPs. By introducing TIMP-mimetic peptides/mini-proteins that reproduce key binding loops to the hydrogel, these bind tightly and specifically to MMPs. Then mild pH/salt shifts, competitor peptides, or cleavable tethers (chemical, photo, or enzyme-triggered) can be used to release the MMPs for analysis. TIMP-mimetic peptides can be made to represent the full, 40 amino-acid N-terminal domain of a natural TIMP. This is the most robust TIMP-mimetic formulation for MMP binding. A minimal inhibitory peptide can be made with 20 amino-acids. This is less expensive, but may require an additional disulfide formation for proper folding. The simplest route is a linear peptidomimetic of 15 amino-acids without disulfides, modified for hydrogel attachment. A TIMP peptide is conjugated to a PEG linker and maleimide, then grafted onto the hydrogel.

Embodiment E incorporates negatively charged sulfated glycosaminoglycans (e.g., heparin) into the hydrogel, or grafts heparin post-polymerization. Proteases like MMP bind to them. MMPs contain positively charged patches (lysine/arginine residues) in their hemopexin and hinge domains that bind negatively charged heparin/heparan sulfate. Three approaches can be used to make these hydrogels.
Approach 1: Heparin-Methacrylate Co-Polymerization: a hydrogel composition is formed by co-polymerizing a heparin derivative bearing polymerizable functional groups with one or more acrylate- or methacrylate-terminated polymers.

In an illustrative embodiment, a heparin-methacrylate intermediate is prepared by reacting heparin with a vinyl-containing anhydride or chloride (for example, methacrylic anhydride or acryloyl chloride) under aqueous or mixed-solvent conditions to introduce pendant unsaturated groups on the heparin backbone. The modified heparin is purified (for example, by dialysis and lyophilization) to obtain a polymerizable, sulfated heparin derivative.

The polymerizable heparin is combined with a hydrophilic crosslinker such as poly(ethylene glycol) diacrylate (PEGDA), multi-arm PEG-acrylate, or equivalent vinyl-terminated polymers in an aqueous medium (e.g., phosphate-buffered saline) together with a free-radical or photoinitiator such as Irgacure 2959, lithium phenyl-2,4,6-trimethylbenzoylphosphinate, or ammonium persulfate/TEMED. The mixture is degassed and polymerized by ultraviolet or visible-light exposure, or by chemical initiation, to form a crosslinked hydrogel network in which sulfated heparin chains are covalently incorporated throughout the matrix.

The resulting hydrogel presents immobilized anionic sulfate and carboxylate groups that enable electrostatic interaction and retention of positively charged or heparin-affine biomolecules, including proteases such as MMPs.
Approach 2: Heparin-Maleimide Post-Grafting: a hydrogel is functionalized with a sulfated polysaccharide such as heparin or a heparin derivative through a thiol-reactive coupling chemistry.

In an illustrative embodiment, heparin is first modified to introduce reactive hydrazide or amine groups by carbodiimide coupling with a bifunctional linker such as adipic dihydrazide, ethylenediamine, or other di-amine or hydrazide compound in a buffered aqueous medium (for example, 2-(N-morpholino) ethanesulfonic acid (MES) buffer) in the presence of a carbodiimide such as 1-ethyl-3-(3-dimethylaminopropyl) carbodiimide and optionally N-hydroxysuccinimide. The resulting hydrazide- or amine-functionalized heparin is subsequently reacted with a maleimide-, vinylsulfone-, or other thiol-reactive moiety, optionally through a PEG spacer, to generate a thiol-reactive heparin derivative.

A hydrogel bearing free thiol groups—such as a cysteine-containing gelatin, thiolated alginate, thiol-modified PEG, or other sulfhydryl-functionalized polymer—is then reacted with the thiol-reactive heparin derivative under mild aqueous conditions to produce a covalent thioether linkage (e.g., Gel-S-Heparin). The reaction may proceed by thiol-maleimide click coupling, thiol-vinylsulfone Michael addition, or other equivalent thiol-reactive chemistry.

The resulting material comprises a hydrogel matrix with covalently grafted, sulfated heparin chains distributed throughout its network. These immobilized heparin domains confer strong anionic charge density and selective affinity for positively charged or heparin-binding biomolecules, including MMPs, growth factors, and cytokines.
Approach 3: EDC/NHS Post-Coupling to Amine Gels: a hydrogel containing free amine groups is functionalized with heparin or another sulfated polysaccharide by carbodiimide-mediated coupling.

In an illustrative embodiment, heparin is activated by reaction of its carboxyl groups with a carbodiimide compound, such as EDC, optionally in the presence of NHS or a water-soluble derivative such as sulfo-NHS, in a buffered aqueous medium (for example, 2-(N-morpholino) ethanesulfonic acid (MES) buffer, pH 5-6). The activated heparin intermediate is then contacted with an amine-functionalized hydrogel, such as gelatin, chitosan, polyethylene glycol amine (PEG-NH₂), or other amine-containing polymer network.

During incubation, covalent amide bonds form between activated carboxyl groups of heparin and amine groups of the hydrogel, yielding a hydrogel matrix with covalently grafted heparin chains. The modified hydrogel retains the anionic sulfate and carboxylate groups of heparin, enabling electrostatic attraction and binding of positively charged or heparin-affine biomolecules, including proteases such as MMPs. Equivalent coupling reagents (for example, DCC/NHS, EDC/HOBt) and buffers or conditions that facilitate carbodiimide-mediated condensation reactions may also be used.

Embodiment F captures MMPs with antibody-functionalized hydrogels. Highly specific antibody-antigen recognition captures target MMP with minimal cross-reactivity. The numerous monoclonal/polyclonal antibodies are distributed on short flexible tethers in a hydrogel; tethers orient the IgG to keep their antigen-binding part exposed. Proper antibody orientation (Fab exposed, Fc anchored) maximizes binding efficiency. This method is highly selective, targeting single MMP isoforms. Antibodies are optimal presented, and there is no substrate or metal interference. Note that antibodies are distributed in moderate density, and not overcrowded. This captures more enzyme, not less. Two principal approaches can be used to incorporate antibodies to hydrogel.

### Approach 1: Protein A/G Scaffold Method

In certain embodiments, antibodies are immobilized within a hydrogel matrix through an intermediate Fc-binding scaffold such as Protein A, Protein G, Protein A/G, or a recombinant or synthetic analog thereof. This approach enables oriented immobilization in which the Fc region of the antibody binds to the scaffold, maintaining the antigen-binding Fab regions exposed for target recognition.

### Phase 1: Immobilization of Fc-Binding Scaffold

In an illustrative embodiment, the Fc-binding scaffold is functionalized with a reactive group capable of forming a covalent linkage to the hydrogel matrix. Exemplary coupling chemistries include thiol-maleimide addition, amine-NHS condensation, azide-alkyne cycloaddition, or carbodiimide crosslinking.

The hydrogel substrate may comprise any polymer bearing complementary reactive groups, such as thiolated PEG, gelatin, alginate, chitosan, or acrylate-terminated polymers. The functionalized Protein A/G (or equivalent) is introduced into the hydrogel precursor solution or contacted with a pre-formed gel under mild aqueous conditions to yield a scaffold-modified hydrogel in which Fc-binding domains are accessible at the gel surface or within its pores.

### Phase 2: Loading of Antibodies

Antibodies specific for one or more target biomarkers—such as matrix metalloproteinases (MMPs)—are contacted with the scaffold-modified hydrogel under conditions favoring Fc-binding. The Fc regions of the antibodies non-covalently associate with the Protein A/G sites, thereby orienting the Fab regions outward for optimal antigen capture. The antibody loading step may be performed with monoclonal, polyclonal, or recombinant antibody preparations. Excess unbound antibody is removed by washing with buffer to yield an antibody-functionalized hydrogel.

In alternative embodiments, Fc-binding scaffolds may be replaced or supplemented by synthetic peptides, engineered proteins, or ligands exhibiting selective affinity for the Fc region of immunoglobulins. Antibody attachment may be stabilized, if desired, by secondary crosslinking (for example, glutaraldehyde, bis-NHS linkers, or photochemical coupling).

The resulting hydrogel contains antibodies immobilized in a defined orientation, providing high binding efficiency and specificity toward their target analytes within biological fluids.

### Approach 2: Fc-Selective Antibody Conjugation via Glycan Oxidation and Click Coupling

### Phase 1: Site-Selective Antibody Modification

In certain embodiments, antibodies are site-specifically functionalized at glycan residues within their Fc region to provide reactive groups for conjugation while preserving antigen-binding activity. In an illustrative embodiment, carbohydrate moieties in the Fc region are oxidized to generate aldehyde groups by reaction with a mild oxidizing agent such as sodium periodate under aqueous conditions. Excess oxidant is removed by buffer exchange or dialysis.

The oxidized antibody (containing Fc-CHO groups) is then reacted with a bifunctional linker bearing a hydrazide, aminooxy, or equivalent aldehyde-reactive group on one terminus and a coupling handle (for example, maleimide, alkyne, azide, or acrylate) on the other. The reaction proceeds through formation of a hydrazone or oxime linkage between the aldehyde groups of the antibody and the reactive hydrazide or aminooxy groups of the linker. The resulting modified antibody contains a reactive end group capable of undergoing subsequent click or Michael-type addition reactions. The product may be purified by desalting, chromatography, or ultrafiltration.

Equivalent strategies that functionalize carbohydrate or peptide residues in regions of the antibody distal from the antigen-binding sites may also be employed.

### Phase 2: Coupling to Reactive Hydrogel

The modified antibody is subsequently coupled to a hydrogel bearing complementary reactive groups. In one embodiment, a hydrogel containing thiol groups is contacted with a maleimide-functionalized antibody under mild aqueous conditions, forming a stable thioether bond. Alternative chemistries, including azide-alkyne cycloaddition, amine-NHS condensation, or photochemical coupling, may likewise be employed depending on the reactive functionality introduced in Phase 1.

After coupling, unbound antibody is removed by washing in buffered saline, yielding a hydrogel with covalently immobilized, orientation-controlled antibodies whose Fab regions remain available for binding target analytes.

Embodiment G uses lab-evolved aptamers or small binding peptides to capture MMPs. These fold into 3D structures that recognize and bind specific MMP surface features (pockets, loops, domains) with high affinity and selectivity. Salt, pH, or temperature changes can release the bound enzymes, or this can be done with competitor oligos/peptides, or photocaged aptamers can be trigger-opened. MMP-8 aptamers are not available from commercial sources at this time, but MMP-1, MMP-9, and MMP-13 aptamers are available, and may be cross-reactive. Embodiments to release bound enzymes include 1) increasing salt in the solution to weaken electrostatic bonds holding the MMP; 2) changing acidity to alter aptamer shape or charges on the enzyme, loosening the bonds; 3) Increasing temperature to destabilize the aptamer's folded structure, releasing the enzyme; 4) Adding DNA, RNA, or peptide that mimics the binding site to solution to induce the MMP to switch from internal to external bonding; 5) Engineering aptamers with a tiny “photocage” that can be broken by a light wavelength, releasing their bound enzymes.

Embodiment H captures MMPs through electrostatic binding on charged functional groups. MMPs have surface charge patches (positive and negative regions) that interact electrostatically with oppositely charged groups in the gel. Unlike specific affinity methods, ion exchange is non-selective but simple and scalable. Most MMPs at pH 7-7.5 are slightly negative, so overall anion exchange is preferred. They have charged patches, such as hemopexin domains that have basic (positive) patches, catalytic domains with variable charges, and hinge regions that are often Both cation and anion exchange in same hydrogel allow multivalent binding. For a multivalent formulation, mix PEGDA (8% w/v), MAETAC (0.5-1% w/v, anion exchange), AMPS 0.5-1% w/v, cation exchange) and Irgacure 2959 (0.1% w/v) in PBS pH 7.4. Photopolymerized, this creates an amphoteric hydrogel with both charge types, that can bind MMPs via multiple charged patches simultaneously.

Embodiment I captures MMPs with PEG chains with terminal oligonucleotide probes (DNA, LNA, or PNA) that selectively hybridize with complementary microRNAs through Watson-Crick base pairing. has a sugar locked in C3′-endo conformation, which gives it higher binding affinity than DNA and makes it nuclease-resistant, and PNA has a peptide backbone and is most nuclease-resistant, with a neutral charge. To design complementary oligonucleotide probes, the microRNAs to bind are analyzed and the reverse complement of nucleotides formed with 5′-amine or thiol modifications. Then the DNA, LNA, or PNA sequences are conjugated to NHS-PEG-acrylate (2-5 kDa), purified by precipitation or desalting, and co-polymerized to yield probe-functionalized gels. Alternatively, oligo-PEG-maleimide conjugates can be grafted to pre-formed thiol-gels via click chemistry. Hydrogels are equilibrated in hybridization buffer prior to use. After collection of the microRNA in the hydrogel, it can be released with a change of temperature or salt contents; these weaken the hybridized strands, and they separate. An embodiment uses a short displacement strand to peel the microRNA off the DNA or DNA-like fragments, called toehold-mediated strand displacement. An embodiment washes the collected microRNAs from the hydrogel with a mild formamide/urea rinse, which weakens the hydrid base pairing and releases the microRNAs.

Embodiment J incorporates positively charged polymers into hydrogels to create electrostatic binding sites for RNA and microRNA, which have negatively charged phosphate backbones (pKa˜1-2, ionized at pH>3). This captures all RNA molecules and RNA-loaded vesicles (exosomes, microvesicles) non-selectively. Hydrogels with positive charges are made, in one embodiment, with a structural backbone of PEGDA and either methacrylated chitosan, PLL-methacrylate, or a cationic monomer. Another embodiment modifies hydrogels after they gel, soaking them in chitosan solution or poly-L-lysine. After use of the hydrogel, the collected, attached RNA and RNA-carrying vesicles are released. In one embodiment salt levels are increased in the hydrogel, which screens electrostatic charges and releases RNA and RNA-carrying vesicles. In an embodiment pH is changed to weaken electrostatic attraction.

Embodiment K captures the exosomes that carry microRNA cargo, instead of free microRNA directly. The antibody-functionalized hydrogel can be an amine gel (for EDC/NHS coupling), a thiol gel (for maleimide click chemistry) or alkyne gel (for azide-alkyne click). Click chemistry ensures antibodies are attached in the correct orientation. Exosomes with particular surface markers preferentially bind to the antibodies. Alternatively, antibodies can be grafted onto already gelled hydrogel by incubation with protin A/G gel. Embodiments can mix antibodies, to capture exosomes expressing a range of markers (like CD63, CD9, and CD81).

A suite of embodiments release antibody-bound exosomes. One embodiment briefly exposes the hydrogel to a reduced pH which loosens antibody binding and releases the entire exosome. This can involve pH-switch (histidine-engineered) antibodies that release at pH˜5.5-6.0. Standard antibodies release at pH 2-3 (too harsh to preserve exosomes.) Histidine-engineered antibodies release at pH 5.5-6.0. Histidine residues (pKa˜6.0) are engineered into antibody-antigen interface. At pH 7.4 the histidine is uncharged and the antibody binds the exosome. At pH 5.5-6.0, histidine protonates and has positive charge, which causes electrostatic repulsion that releases antibodies. An embodiment adds an excess of the tetraspanin's extracellular-loop peptide (in large excess, 10-100 μM) which displaces the entire vesicle, or adds neutralizing Fabs (antibody fragments that block vesicle attachment) thereby released the entire vesicle. These are synthetic peptides mimicking the CD63/CD9/CD81 epitope that antibody recognizes. The competing peptide binds antibody and displaces the entire exosome. An embodiment uses high ionic strength or chaotropes to disrupt antibody binding, which involves electrostatic and H-bond interactions. 0.5-2 M NaCl disrupts ionic interactions, releasing ˜60-80% of exosomes. 2-4 M urea disrupts H-bonds and hydrophobic interactions, releasing ˜80-90% of exomes. An embodiment uses high ionic strength or chaotropes to disrupt antibody binding, which involves electrostatic and H-bond interactions. 0.5-2 M NaCl disrupts ionic interactions, releasing ˜60-80% of exosomes. 2-4 M urea disrupts H-bonds and hydrophobic interactions, releasing ˜80-90% of exomes. An embodiment adds a reducing agent to cleave the disulfide bridge that forms a bond between PEG and the antibody. The reducing agent may be TCEP or DTT. An embodiment adds a protease not present in sample that cleaves the peptide linker and releases both antibody and exosome. Such proteases include TEV protease. Of all the exosome release embodiments, high salt and urea are the simplest, while disulfide and protease cleavage recover the highest fraction of intact exosomes.

Table 3 compares the different release mechanisms.

TABLE 3

| | | | | | | | Exosome | |
|---|---|---|---|---|---|---|---|---|
| Method | pH | Temp | | Time | | Recovery | Integrity | Complexity |
| pH-switch | 5.5-6.0 | 25° | C. | 15-30 | min | 70-85% | Good | Medium (needs |
| (His-Ab) | | | | | | | | engineered Ab) |
| Peptide | 7.4 | 37° | C. | 1-4 | h | 50-70% | Excellent | Medium (peptide |
| competition | | | | | | | | synthesis) |
| Neutralizing | 7.4 | 25-37° | C. | 1-2 | h | 60-80% | Excellent | Medium (Fab |
| Fab | | | | | | | | generation) |
| High salt | 7.4 | 25° | C. | 30-60 | min | 60-80% | Good (need | Low |
| | | | | | | | desalting) | |
| Urea | 7.4 | 25° | C. | 30 | min | 80-90% | Moderate | Low |
| | | | | | | | (dialyze fast) | |
| Disulfide | 7.4 | 37° | C. | 30-60 | min | 80-90% | Excellent | High (linker |
| cleavage | | | | | | | | design) |
| Protease | 7.4 | 30° | C. | 2-4 | h | 85-95% | Excellent | High (linker |
| cleavage | | | | | | | | design) |

An embodiment uses antibodies against ESCRT-related proteins to capture microRNA containing exosomes. ESCRT (Endosomal Sorting Complex Required for Transport) are multi-protein complexes that drive exosome biogenesis by budding vesicles from endosomal membranes. These proteins are part of the vesicle-forming machinery and often remain associated with mature exosomes, making them universal markers. In an embodiment cell adhesion and fusion proteins, such as integrins, ICAM-1, and lamp proteins, involved in exosome docking, are used as markers for antibodies to capture microRNA containing exosomes. Integrins are a/B heterodimers that mediate exosome-cell interactions. They are present on most exosomes at varying levels. LAMP proteins are present on exosomes from multivesicular body pathway, such as the pancreas.

In an embodiment heat shock proteins (HSPs) found on external membranes of exosomes are used for antibody generation, enabling the antibodies to capture them. Either the whole heat shock protein is used, or a piece of the HSP unique to a particular protein's identity.

An embodiment binds microRNA inside exomes by opening or permeabilizing the vesicles to render the internal RNA accessible, then using any of the previous embodiments that use probes attached to hydrogels that capture, collect, concentrate, then release on command the microRNA. An embodiment incorporates gentle detergent molecules (saponin, digitonin) into the hydrogel; these are gradually released to puncture vesicles. An embodiment makes hydrogels with local areas of elevated salt or altered acidity to stress vesicle membranes and make them permeable. An embodiment captures the entire exosomes in the hydrogel, then cycles the hydrogel temperature down and up to stress and fracture membranes to expose the RNA.

Embodiment L polymerizes the hydrogel in the presence of a urea template. The urea creates shape-specific cavities with complementary hydrogen bonding sites. After the urea is washed out, urea from saliva can be bound via shape and chemical recognition. To built a urea-based hydrogel, structural monomers are mixed with functional monomers such as methacrylic acid that H-bonds with urea, as well as crosslinkers. The functional monomer holds the urea template in place with hydrogen bonds at either ends to a carboxylic acid group of methacrylic acid: COOH—H₂N—CO—NH₂ (the urea)—COOH. The urea is pre-complexed with methacrylic acid, which locks in the desired spatial configurations. The urea complex is added to the monomers in 3-10% w/v, with a catalyst and initiator (ammonium persulfate). After polymerization, the hydrogel has urea molecules trapped inside, with the polymer network defining cavity shapes. Washing out urea reveals imprinted sites inside the hydrogel.

Embodiment M immobilizes urease enzyme in a negatively charged hydrogel. Urease converts urea to ammonia in situ, and ammonia protonates to NH₄⁺. Negatively charged groups (—SO₃⁻, —COO⁻) electrostatically trap NH₄⁺ and concentrate this signal. Eluting and measuring ammonia is an indirect urea quantification. A negatively charged hydrogel with urease is made with PEGDA or acrylamide and an anionic monomer, with urease enzyme added. After polymerization, the soft gel has entrapped urease. After use, captured NH₄⁺ is eluted with high salt or mild base, then measured colorimetrically or electrochemically to indirectly quantify the original urea concentration. This approach provides signal amplification through enzymatic conversion combined with electrostatic concentration.

Embodiment N polymerizes a hydrogel around cortisol, using the cortisol as a template. After polimerization, the cortisol is washed out, leaving behind cortisol-shaped cavities in the hydrogel matrix. This is achieved by forming a monomer mixture: acrylamide in bulk, methacrylic acid (which is the H-bond donor/acceptor) and a crosslinker (EGDMA or BIS). Cortisol is dissolved in an ethanol/water blend to make the cortisol template. The monomer mixture (˜10-20% w/v) is added to the cortisol template. To polymerize, ammonium persulfate is added followed by TEMED, which triggers the acrylate or acrylamide groups in the monomers to connect and form a polymer network (the hydrogel). Finally cortisol is extracted with repeated washes of ethanol:water (50:50); this can be confirmed when laboratory analysis by liquid chromatography or UV spectroscopy no longer detects any cortisol in the wash solution. Polymerization can also be triggered by a photoinitiator, usually UV or blue light, that releases free radicals that link monomers and crosslinkers into a hydrogel network. After Hydrogel Ball use, the housing is opened, and the hydrogel inside is placed in a device container.

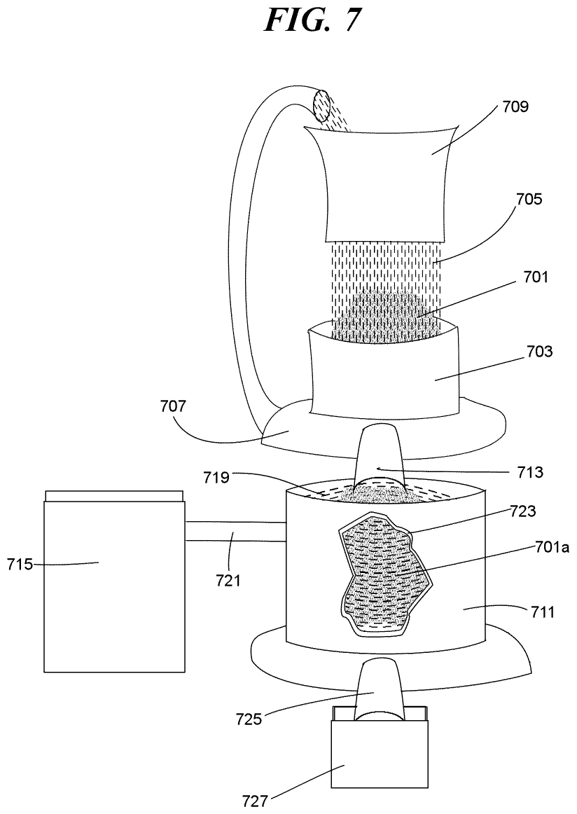

FIG. 7 illustrates a hydrogel 701 in device rinse container 703. Hydrogel 701 is rinsed with PBS rinse 705 composed of 150 mM NaCl and 0.01% Tween-20, to remove loosely bound proteins. The device container 703 rests on a reflow unit 707 that cycles the PBS rinse 705 into wash unit 709 multiple times to stream the PBS rinse 705 on the hydrogel 701. After the hydrogel is rinsed, hydrogel 701a is moved into elution station container 711, or rinse container 703 with hydrogel 701a is moved into elution station 711. Hydrogel 701a is visible through a cut-away 723 of elution station 711. Hydrogel 701 is visible through a cutaway in elution station container 711. Rinse fluid residue (not visible) from rinse container 703 is passed through conduit A 713 into elution station 711. Elution buffer container 715 contains elution buffer 719; elution buffer 719 passes through conduit B 721 into elution buffer station 711. Cortisol is small and diffuses quickly, so 5 to 10 minutes of soaking hydrogel 701a in elution buffer 719 allows cortisol to migrate out of the hydrogel 701a's pores. Elution buffer 719 may be one of the following: 1) 1-10 μM cortisol in PBS for 5-10 minutes; 2) 10-20% ethanol in PBS for 5-10 minutes; 3) 100 mM glycine-HCl for 60 seconds, immediately neutralized in 10 times the volume of PBS; 4) 0.5-1.0 M NaCl in PBS for 5-10 minutes. After the period of soaking, eluate (not visible) passes through conduit C 725 into sample container 727. In an embodiment the user pipettes a volume of the eluate onto a conjugate pad that contains a commercially available cortisol lateral flow immunoassay (LFIA). The pad contains antibodies against cortisol. Antibody complexes can bind to either free cortisol in the sample or immobilized cortisol on the pad line. The more cortisol in the sample, the less the pad line cortisol can bind to the antibodies—and the weaker the line appears. Another embodiment uses ELISA to determine cortisol volume. The eluate is put in cocortisol-specific antibody wells, and an enzyme added that produces a color proportional to cortisol concentration. Following use of the Hydrogel Ball and determination of cortisol levels, embodiments with molecularly imprinted hydrogels with cortisol-shaped cavities can be easily reused. Cortisol and saliva residues must be fully removed to keep imprint sites accessible for reuse. This cleaning takes place during the initial rinsing phase, when hydrogel 701 is rinsed with PBS rinse 705. If cortisol binding drops, a glycine-HCl blend, or 10 mM Na₂CO₃, serve as anti-fouling agents. The hydrogel is rinsed with these for 2-3 minutes, then neutralized in PBS.

An embodiment that binds cortisol immobilizes anti-cortisol IgG/Fab in a hydrogel with an orientation that leaves the binding site exposed. Fc-selective covalent immobilization locks the IgG in place. In certain embodiments, the immobilized anti-cortisol antibody hydrogel is regenerable and reusable for multiple capture-release cycles by competitive or mild solvent elution followed by neutralization and re-equilibration, while maintaining functional binding capacity within acceptable limits.

An embodiment immobilizes CBG (corticosteroid-binding globulin, the native cortisol carrier) into a hydrogel, which uses receptor-decoy capture for high-affinity cortisol binding. In certain embodiments, a hydrogel bearing CBG is regenerable and reusable for multiple capture-release cycles by competitive or mild solvent elution followed by neutralization and re-equilibration, while maintaining functional binding capacity within acceptable limits.

An embodiment grafts β-cyclodextrin to hydrogel to create hydrophobic pockets. Cortisol forms complexes with β-cyclodextrin. An embodiment generates hydrophobic regions in hydrogel that preferentially partition cortisol.

There are many other salivary biomarkers that can be bonded and captured in a hydrogel using agents grafted into the gel (plus typical release options). Some are shown in Table 3. Binding agents can be specific, to isolate particular biomarkers, or general, when the biomarker needs to be concentrated, but not isolated from other molecules.

Embodiments graft binding agents to hydrogels with generic chemistries, and additional factors listed in Table 4. Generic chemistries are EDC/NHS (—COOH↔—NH₂), maleimide-thiol, click (azide-alkyne), and (desthi)biotin-streptavidin.

TABLE 4

| Biomarker | Specific Bonding | General Bonding | Grafting | Release |
|---|---|---|---|---|
| C-reactive protein | Anti-CRP antibodies | Cation/anion exchange | Phosphocholine (PC) | Ca²⁺ removal for PC |
| (CRP; pentraxin) | (IgG/Fab) | (CRP charge varies | headgroup ligands | systems, or PC |
| | | with pH) | (immobilized PC or | competitor; mild |
| | | | PC-mimetic) bind | salt/pH for antibodies |
| | | | CRP's Ca²⁺-dependent | |
| | | | site. | |
| Calprotectin | Anti-calprotectin | Ion-exchange (often | Generic chemistries | salt/pH; competitive |
| (S100A8/S100A9 | antibodies, also | acidic proteins at | | peptide; standard |
| heterodimer; metal- | RAGE-domain or | neutral pH) | | antibody elution |
| binding) | peptide mimetics | | | |
| Alkaline phosphatase | Anti-ALP antibodies | Anion exchange at pH | Tethered via a spacer | Competitive inhibitor |
| (ALP; Zn/Mg- | (choose isoform if | 7-8 (many ALPs are | or phosphate-mimic | in eluate; salt/ |
| dependent hydrolase) | needed) | basic but have | ligands | pH shift. |
| | | negative patches) | | |
| Lactate | Anti-LDH antibodies | Ion-exchange | Cofactor-site ligands: | NAD⁺ or oxamate |
| dehydrogenase | | | immobilized NAD⁺/ | (pyruvate analog) as |
| (LDH; NAD⁺- | | | ADP analogs or dye- | competitor; salt for |
| dependent) | | | ligands that mimic | dye-ligand systems. |
| | | | nucleotide binding | |
| Aspartate | Anti-AST/anti-ALT | Ion-exchange | Substrate/transition- | Substrate analog |
| aminotransferase & | antibodies | | state mimics | competitor; salt/pH |
| Alanine | | | (immobilized α-keto | |
| aminotransferase | | | acid or amino acid | |
| | | | analogs via a spacer) | |
| Hepatocyte growth | Anti-HGF antibodies | Weak cation exchange | Heparin/heparan- | High-salt (0.5-1.0M |
| factor | or c-Met receptor | (HGF has basic regions | sulfate grafts, | NaCl) from heparin |
| | domain | that interact with | immobilized. GAG- | gels; competitive |
| | | GAGs) | modified hydrogel | heparin; mild pH shift. |
| Cytokines | Monoclonal antibodies, | Some cytokines show | Heparin/heparan- | mild acid (pH 5.5-6.0) |
| IL-17 | Receptor-mimetic | heparin/GAG affinity | sulfate grafts | for receptor/antibody |
| | capture: immobilized | screen heparin- | | systems; salt or |
| | IL-17RA/RC | modified gels, but | | competitor peptide |
| | ectodomains | expect variability. | | |
| IL-23 | Monoclonal antibodies, | | Immobilize Protein | |
| | Decoy receptors IL- | | A/G (covalent), load | |
| | 23R/IL-12Rβ1 | | antibodies Fab out. | |
| IL-1β | Monoclonal antibodies, | Ion-exchange for pre- | Immobilize ectodomains | |
| | Decoy receptors IL- | concentration. | or covalently attach | |
| | 1R1 | | antibodies | |

All numbers expressing quantities, percentages, or proportions are to be understood as being modified by the term “about” unless otherwise indicated. Ranges may be expressed as from one value to another, and such ranges are intended to include all subranges therebetween. The disclosure of individual values serves as a disclosure of the entire range and all subranges.

As used herein, the singular forms “a,” “an,” and “the” include plural referents unless the context clearly dictates otherwise. The term “comprising” is intended to be open-ended and includes the term “consisting essentially of” and “consisting of.” All technical and scientific terms used herein have the same meaning as commonly understood by one of ordinary skill in the art.

The scope of the invention should not be limited by the particular embodiments described herein, but should be defined only by the appended claims and their equivalents. Where a means or step is recited in the claims without specifying structure, it is intended that the recitation be interpreted broadly, consistent with 35 U.S.C. § 112(f)(pre-AIA § 112, sixth paragraph).

All publications, patents, and patent applications cited in this specification are hereby incorporated by reference in their entirety for all purposes as if each individual publication or patent were specifically and individually indicated to be incorporated by reference.

Accordingly, the invention is not to be limited except as by the appended claims.

1. A Hydrogel Ball comprising: a flexible housing having at least one layer configured to contain a hydrogel therein; the flexible housing including a plurality of holes, the holes comprising perforations, apertures, or pores sized to permit fluid exchange between the hydrogel and an external environment while retaining the hydrogel; the hydrogel containing a fluid, the fluid being capable of flowing out of and into the hydrogel during contact with the external environment; the Hydrogel Ball being sized and configured for placement in a mouth of an animal, wherein saliva present in the mouth mixes with the fluid and biomarkers in the saliva are absorbed and retained within the hydrogel.

2. The Hydrogel Ball of claim 1, wherein the hydrogel comprises a plurality of antibodies configured to bind specific biomarkers captured in the hydrogel.

3. The Hydrogel Ball of claim 1, wherein the hydrogel comprises a plurality of hydroxamate groups tethered to flexible polyethylene glycol linkers, the hydroxamate groups being configured to chelate zinc atoms of matrix metalloproteinase (MMP) collagenases present in the saliva, thereby immobilizing the MMP collagenases in the hydrogel.

4. The Hydrogel Ball of claim 1, wherein the hydrogel comprises a plurality of tethered peptides selected from the group consisting of collagen-like peptides and tissue inhibitor of metalloproteinase-mimetic peptides, the tethered peptides being configured to bind MMP collagenases present in the saliva, thereby immobilizing the MMP collagenases.

5. The Hydrogel Ball of claim 1, wherein the hydrogel contains a plurality of binding agents selected from the group consisting of:
(a) antibodies against one or more vesicle surface markers, the markers comprising CD63, CD9, CD81, ESCRT proteins, integrins, ICAM-1, LAMP proteins, or heat shock proteins, the antibodies configured to capture exosomes containing nucleic acids that are some of the plurality of biomarkers in the saliva; and
(b) nucleic acid probes tethered to the hydrogel, the probes comprising DNA, Locked Nucleic Acid, or Peptide Nucleic Acid, the probes configured to form complementary hybrids with nucleic acids contained in exosomes or released therefrom.

6. The Hydrogel Ball of claim 5, wherein one or more exosomes captured in the hydrogel are permeabilized to expose internal nucleic acids for hybridization with the tethered probes.

7. The Hydrogel Ball of claim 1, wherein the hydrogel comprises one or more binding agents selected from the group consisting of: (a) glycosaminoglycan chains configured to capture proteases present in the saliva; (b) ion-exchange groups configured to capture proteins present in the saliva; and (c) nucleic acids, amino acids, or peptides generated through in vitro evolution, configured to fold around and bind enzymes or nucleic acids present in the saliva.

8. The Hydrogel Ball of claim 7, wherein the binding agents are present in combination to capture multiple different classes of biomarkers simultaneously.

9. The Hydrogel Ball of claim 1, in which the hydrogel is imprinted with a plurality of cavities generated by polymerization; the plurality of cavities concentrate biomarkers in the saliva.

10. The Hydrogel Ball of claim 1, wherein one or more enzymes are immobilized in the hydrogel, the enzymes being configured to convert organic compounds present in saliva into forms that bind to the hydrogel.

11. The Hydrogel Ball of claim 1, wherein the mechanism that releases biomarkers from the hydrogel comprises a reversible binding strategy selected from the group consisting of: (a) cleavage of one or more cleavable linkers; (b) competitive elution by molecules having a shape, charge, or binding motif recognized by target proteins; (c) modulation of environmental conditions, including a pH shift, a change in salt concentration, a temperature change, or exposure to a mild chaotrope; (d) chelation control through reversible sequestration of metal-dependent binding interactions; and (e) displacement of nucleic acids by introduction of complementary oligonucleotides.

12. A Hydrogel Ball comprising:
a flexible housing comprising at least one layer configured to contain a superabsorbent or swellable material;
the flexible housing being adapted to permit exchange of fluids between the superabsorbent or swellable material and an external environment, wherein the fluids entering the superabsorbent or swellable material contain one or more biomarkers;
the Hydrogel Ball being sized and configured for placement in a bodily cavity of an animal;
after use, the superabsorbent or swellable material is removed from the flexible housing and deposited in a container;
an elution buffer is added to the container to soak the superabsorbent or swellable material, thereby releasing the one or more biomarkers and generating an eluate.

13. The Hydrogel Ball of claim 12, wherein the superabsorbent or swellable material comprises a superabsorbent hydrogel modified with one or more binding agents selected from the group consisting of: antibodies, peptides, hydroxamate groups, glycosaminoglycans, nucleic acid probes, or ion-exchange groups, the binding agents being configured to capture the one or more biomarkers present in fluids entering the hydrogel.

14. The Hydrogel Ball of claim 12, wherein the one or more biomarkers are selected from the group consisting of: proteases, nucleic acids, exosomes, or proteins.

15. The Hydrogel Ball of claim 12, wherein the release of the one or more biomarkers from the superabsorbent or swellable material is triggered by one or more of: cleavage of a cleavable linker, competitive elution, a change in pH, salt concentration, or temperature, or addition of a chelator compound.

16. The Hydrogel Ball of claim 12, wherein the eluate is analyzed to determine the presence or concentration of one or more biomarkers.

17. A method of collecting salivary biomarkers from an animal, the method comprising: placing in the oral cavity of the animal a Hydrogel Ball composed of a flexible housing with a superabsorbent hydrogel disposed inside the flexible housing; allowing fluid contained in the superabsorbent hydrogel to exit the Hydrogel Ball and enter the oral cavity; allowing the fluid from the superabsorbent hydrogel to mix with saliva present in the oral cavity; reabsorbing the mixture of the saliva and the fluid into the superabsorbent hydrogel; and capturing some of the plurality of salivary biomarkers in the superabsorbent hydrogel.

18. The method of claim 17, wherein capturing some of the plurality of salivary biomarkers in the superabsorbent hydrogel comprises binding agents incorporated in the superabsorbent hydrogel, the binding agents being selected from the group consisting of: antibodies, hydroxamate groups on polyethylene glycol linkers, tethered peptides, glycosaminoglycan chains, ion-exchange groups, or nucleic acid probes.

19. The method of claim 17, further comprising releasing some of the plurality of biomarkers from the superabsorbent hydrogel by a mechanism comprising one or more of: cleavage of a cleavable linker, competitive elution, modulation of environmental conditions including a pH shift, salt concentration change, temperature change, or exposure to a mild chaotrope, chelation control, or displacement of nucleic acids by complementary oligonucleotides.

20. The method of claim 17, wherein the Hydrogel Ball is sized and configured to be held in the oral cavity of a mammal selected from the group consisting of dogs, cats, horses, goats, sheep, or humans.
